(19) United States
(12) Patent Application Publication
(10) Pub. No.: US 2007/0048765 A1
(43) Pub. Date: Mar. 1, 2007

(54) Use of genetic information for identity authentication

(76) Inventor: Fredric David Abramson, Gaithersburg, MD (US)

Correspondence Address: Fredric D. Abramson;AlphaGenics, Inc., 9700 Great Seneca Highway, Rockville, MD, 20882, US

(21) Appl. No.: 11/466,499

(22) Filed: Aug. 23, 2006

Related U.S. Application Data
(60) Provisional application No. 60/596,021, filed on Aug. 24, 2005.

(51) Int. Cl. C12Q 1/68; G06F 19/00

(52) U.S. Cl. 435/6; 702/20

(57) ABSTRACT

This invention incorporates genetic information about an individual for use in verifying said individual's identity in transactions where improper identity, identity theft or other use of false identifying information creates financial, personal or other risks. The invention utilizes secure methods wherein an individual's genetic identity is stored by an authenticating service, which provides a duplicate of that genetic identity listing to the individual. The list of the person's genes is randomly sorted without regard to alphabetic or other ordering systems, and the list can be re-randomized at will. A third party can request the individual to supply the specific identity of one or more genes found in one or more specific locations on that list, and that third party can then independently verify with the authentication service that the identity of the genes provided by the person does in fact correspond to the identity of the genes the person is authorized to provide, thereby authenticating the identity of said person. The individual has the ability to create his or her own authentication system by providing a subset of the list to another party, where the subset list is randomized by the individual using software designed for such purpose. This allows the individual to authorize another person to take some action that involves the party to whom the subset list was given. An example is giving permission for someone to pick up a child at day care, where the individual granting permission provides the randomized list to the day care center, and the person picking up the child must properly provide the identity of the genetic keys in accordance with what is contained in the day care center list.

### CROSS-REFERENCE TO RELATED APPLICATIONS

This application claims priority from U.S. Provisional Application No. 60/596,021, filed on Aug. 24, 2005, which is hereby incorporated by reference in its entirety.

### BACKGROUND OF THE INVENTION

1. Field of the Invention

The present invention generally relates to identity security and identity authentication, and more particularly, is concerned with a system and method for using a person's genetic profile for protect and verify their identity in various types of transactions and situations.

2. Description of the Related Art

The problem of identity theft has become a significant factor with on-line retailing becoming a dominant economic force. “The Federal Trade Commission says that ID theft cost U.S. businesses and financial institutions nearly $48 billion in 2003.” (1) “The New Face of Identity Theft” CFO-IT, Spring 2005, at 23. Not only does it rob individuals of their security, and impose great hardships upon them when their identity is stolen, on-line retailers are increasingly susceptible to a variety of identity scams, which are becoming more sophisticated and difficult to detect. The number of fraudulent orders to some online retailers is rising, in some cases to 30%, with actual fraud losses rising to half a percent. (2) “At Online Stores, Sniffing Out Crooks Is a Matter of Survival”, The Wall Street Journal, Aug. 4, 2005, Page A1. Online fraud was over $437 million in 2003, with the median consumer loss being $228. The web savvy 18-39 age group topped the victims with 54 percent of the total loss. (3) “Online Fraud Losses Hit $437M”, http://www.clickz.com/stats/sectors/finance/article.php/3303041, Ryan Naraine, Jan. 23, 2004. U.S. Online retail sales were $21.1 billion in Q2 2005, up 26% from a year ago. (4) U.S. Department of Commerce, August 2005. The identity theft threat is growing partially because of the increased involvement of organized crime groups around the world. (5) “Phishing, Inc.” eWeek, Mar. 7, 2005.

A variety of electronic and biometric means are being created to address this problem. These include such things as sending electronic fingerprint images, images of a person's blood vessel arraignment in the palm, voice prints, iris scans, and other means.

Discussion of using DNA as a screening device are hindered by the cost of repeated testing combined with the long turn-around time between the taking of a sample and the test results. There is also significant public resistance because of the delicate and confidential nature of a person's genes, particularly those associated with disease. One concern is that disease-related genetic information will find its way into the hands of parties who would misuse the information.

Another area of identity verification is biometrics. Biometrics is a priority because it capitalizes on our physical individuality. Methods using fingerprints, hand geometry, palm blood vessel patterns, retinal scans and voice ID are all being developed for commercial application. Each has problems, however, for use in the electronic environment. Voice identification, which can be used on telephones, suffers from having error rates as high as 5 to percent. (6) “A Touchy Subject”, CFO-IT, Spring 2005.

On-line fraud is not the only problem this invention solves. It can lower the risks in non-face-to-face banking transactions, securities transactions and corporate money transfers. It can be used to verify that a person using a company's computer internally is appropriately authorized to do so. It can uniquely be used to identify person's having permission to pick-up children at day care centers, thereby lowering the risks of child abuse and kidnapping. It can be used to verify permitted transmittal of electronic medical records in compliance with HIPAA.

A variety of electronic and biometric means are being created to address the problem of identity theft and fraud. These include such things as sending electronic fingerprint images, images of a person's blood vessel arraignment in the palm, voice prints, iris scans, and other means. Discussion of using DNA as a screening device are hindered by the cost of repeated testing combined with the long turn-around time between the taking of a sample and the test results. There is also significant public resistance because of the delicate and confidential nature of a person's genes, particularly those associated with disease. One concern is that disease-related genetic information will find its way into the hands of parties who would misuse the information.

This invention utilizes non-disease genetic information from an individual in a new way to authenticate that person's identity in on-line transactions, as well as other types of transactions. The invention will be used by people whose identity is to be authenticated by having a one-time genetic test performed to identify the genes.

The number of genes to be screened can vary. As the number increases, the number of permutations increases. In the preferred embodiment, 200 genes are used in the basic screen.

It can readily understood by those schooled in the art that the number of genes to be screened need only satisfy a minimum such that the number of permutations is sufficiently high to avoid random duplication and to deter attempts to defeat the system.

Then, the preferred embodiment further uses subsets of 6 of 200 genes, which provides over 82 Billion authentication combinations. If 8 genes are used, 55 trillion combinations are possible, which are enough to discourage random or systematic attempts to deceive a party.

It should be readily understood by those schooled in the art that the number of genes in the subset can be any number that is less than or equal to the number tested, and need be a sufficient number to reliably verify the identity of the person in the transaction.

The person has his/her (200) genes identified.

The person receives the test results in a specially coded and encrypted device accompanied by a hard copy.

The ordering of the 200 genes sent to the person is randomized. So, for example, consider the gene for eye color in two different people. One person has a ‘blue’ gene, the other a ‘brown gene’. The first person's gene order has the ‘blue’ gene at slot number 39. The second person's gene order has the ‘brown’ gene at slot number 148. By slot we mean the position of the gene from the top or starting point of the list.

Here is an example of how the invention would work in an on-line purchase.

The on-line vendor queries the prospective customer during the transaction and asks that the person specifically identify or ‘name’ the genes at certain places in the rank order.

For example, if five genes are used, the vendor specific the numbers of the genes requested, for example 34, 78, 121, 143, 187 using software provided by the host, who is a trusted intermediary.

The prospective customer provides the vendor the names of the five genes on his/her list that correspond to these ordered numbers.

The vendor then sends to the host the five numbers and genes identified by the prospective customer.

The host then compares the numbers and associated genes with its internal file that contains the authenticating buyer's information and corresponding random list.

If the person matches the five genes, the vendor is told the person is authenticated.

If the person matches two or less of the genes, the person is not authenticated.

If the person matches 3 or 4 genes, the vendor may decide to deny the transaction or submit another authenticating query.

Another scenario involves picking up children at day-care illustrates how this invention would be used to verify that a third-party is permitted to pickup the child. In 1999, there were 7.9 million preschoolers and 1.9 million gradeschoolers in day-care. In this scenario, the mother has her genetic profile performed and receives both a randomized gene listing and/or an ordered gene listing. She then can select a subset of the genes, say 40, and give that subset listing to the day care provider. The subset listing would also be randomized using software provided with the person's profile, where this randomization is independent of the mother's randomization. What this means is that the mother's “taste” gene may be in location 27 in her list, but in location 4 in the day-care center's list.

One day it is necessary for her husband, who the day care provider has never met, to pick up the child. Or an uncle or aunt or neighbor. The mother gives the husband the order number and names of, say, 4 genes: i.e., gene 9=PAX, gene 17=VEGF, gene 24=BAS and gene 38=CRC. The husband person must correctly provide these four order numbers and the corresponding gene names to be permitted to pick up the child.

The day care center will be able to utilize software provided by AlphaGenics to keep track of the gene listings associated with each child. The software can also produce the randomized sub-list queries used to authenticate a permitted pickup. The software can also default into layers or levels of follow-up action in the event invalid authentication occurs, including the possibility of phoning the police for intervention assistance.

The subset listing can be changed at the parent's desire, or on a regular calendar basis. The frequency of changes can be programmed into software or driven by some other means. This ensures the list does not become stale, and also responds to changes in the parent's lives, such as divorce, etc.

Another authenticating use is in corporate or large institutional environments using many computers, where potentially confidential information is at risk to exposure or misuse. One of the challenges is to ensure that the person logging onto a computer is in fact the person authorized to use that computer. Password protection helps, but is far from secure for many reasons. Use of biomarkers such as fingerprints and virus scans are effective, but often costly to implement.

A more cost effective way of handling the problem is to have the employee provide the employer his/her gene listing (with appropriate privacy considerations and protections). The employer then randomizes the list, and gives the employee a copy of the randomized list. Then, when the employee logs onto a computer or workstation, the final authentication step would be to properly and correctly identify a genetic subset selected from the randomized list. Failure to properly authenticate oneself would automatically lock the keyboard/workstation, and enable further action by the employer. Because this system is software driven and has high flexibility and adaptability, it can be implemented at low cost.

This invention can be considered a type of variable social security number. If a person should have their SkyGene lost or stolen (or the hard copy list lost or stolen), AlphaGenics will immediately re-issue a new device, with a new randomized ordering of the person's genes. The number of ordered combinations of 200 genes is astronomical. To illustrate, Microsoft Excel can only compute the number of combinations of 170 genes, which is 7.3 E+306. This is 7.3 followed by 306 zeros. (7,300,000,000,000,000,000,000,000,000,000,000,000,000,000,000,000,000,000,000,000,000,000,000,000,000,000,000,000,000,000,000,000,000,000,000,000,000,000,000,000,000,000,000,000,000,000,000,000,000,000,000,000,000,000,000,000,000,000,000,000,000,000,000,000,000,000,000,000,000,000,000,000,000,000,000,000,000,000,000,000,000,000,000,000,000,000,000,000,000,000,000,000,000,000,000,000,000,000,000,000,000,000).

Thus, although the identity of the persons genes doesn't change, the number of combinations is more than sufficient. And because only a few of the genes are used to authenticate a person, even on-line snooping of a series of transactions by a holder of this invention will fail to reveal sufficient information to successfully steal the person's genetic identity.

In addition, because the gene patterns are randomized for each person, and only a subset of genes is provided the vendor, no vendor can accumulate sufficient transactions to decode any person's pattern. Additional protection comes from the host's ability to automatically track each vendor's transaction history, which would alert us if a potentially compromising set is identified. In such cases, a new randomized gene list will automatically be provided the customer. With 200 genes and 5 genes per validation, the vendor would have to accumulate a very large number of transactions to approach a reasonably useful copy of a person's profile.

This invention utilizes non-disease genetic information from an individual in a new way to authenticate that person's identity in on-line transactions, as well as other types of transactions. The invention involves the person whose identity is to be authenticated having a one-time genetic test performed to identify socially relevant, non-disease genes. Virtually any number of genes can be used for this process, though the greater the number, the more reliable the authentication. The best mode is considered to involve between 100 and 200 genes. As will be described later, adding genes above 200 provides little added value as merely taking subsets of 6 of 200 genes provides over 82 Billion combinations. Using only 8 genes provides 55 trillion combinations, which are enough to discourage random or systematic attempts to deceive a party.

While this invention uses non-disease information, it is obvious to those schooled in the art that any gene can be used, regardless of its function. It is also obvious that the number of genes involved can be greater than 200 or less than 100. The major factors in determining the precise number of genes is the cost of testing, sensitivity of the information to the individual (some people don't want to know if they have bad genes), and other common factors.

It is also obvious to those schooled in the art that the actual coding for any segment of DNA can be used without regard to whether the segment has any functional (i.e., gene) identification. When DNA segments are is used, the identity of single nucleotide polymorphisms in the segment can be readily identified.

Another factor that is obvious to those schooled in the art is that each gene must be polymorphic. This means that there must be more than one version of the gene in the population. The reason is obvious: if there is only one version of a gene, then everyone has it and it can't be used to differentiate one person from another. So for example, the genes for eye color are known to vary. For purposes of this invention, we define a gene as the genetic code residing in a specific location on a chromosome, where variations in the DNA sequence can be found within for that gene in the human population, these variations being referred to as variants or polymorphs or mutations, depending on the usage.

It is obvious to those schooled in the art that when DNA segments are used, the length of each segment and the location of each segment must be such that a sufficient number of variants can be found.

Each person should be tested for the same 200 genes. While it may be possible for different genes to be tested and identified, this would increase the storage and computational requirements at the retailer level, which are likely to make the process too costly to implement in a practical fashion.

The invention works as follows:
(1) The person has his/her (200) genes identified.
(2) Each person is tested for the variants in the same 200 genes.
(3) The person receives the test results in electronic format, in a specially coded and encrypted device, with a hard copy.
(4) The device need not be encrypted.
(5) The information can be provided in any electronic form, including via the Internet.
(6) The ordering of the 200 genes sent to the person is randomized, using a random number generator or other comparable method. So, for example, consider the gene for eye color. One person has a ‘blue’ gene, the other a ‘brown gene’. [for purposes of this invention, heterozygous configurations can be considered a unitary variant or the gene for each pair can be listed separately]. The first person's gene order has the ‘blue’ gene at slot number 39. The second person's gene order has the ‘brown’ gene at slot number 148. By slot we mean the position of the gene from the top or starting point of the list.
(7) The person conducts a commercial or other transaction that requires identity authentication.
(8) The party wishing to authenticate sends a query to the person, by electronic, voice or any other communication means.
(9) The query asks the person to specifically identify or ‘name’ the genes at certain places in the rank order.
(10) For example, if five genes are used, the party provides the numbers of the genes requested, for example 34, 78, 121, 143, 187.
(11) The authenticating person sends back to the party the names of the five genes on his/her list that correspond to these ordered numbers.
(12) The party then sends to the host the five numbers requested and the genes provided by the authenticating person.
(13) The host then compares the numbers and associated genes with its internal file with the authenticating person's information.
(14) If the person matches the five genes, the party is told the person is authenticated.
(15) If the person matches two or less of the genes, the person is not authenticated.
(16) If the person matches 3 or 4 genes, the party may decide to deny the transaction or submit another authenticating query.

If the authenticating party should have the device lost or stolen, the host will re-issue a new device, with a new genetic ordering of the person's genes. Thus, although the identity if the genes themselves don't change, the number of ordered combinations grows to an astronomical number as the number of genes used increases. A mere 15 genes can produce over a billion combinations.

Because only a few of the many genes are used to authenticate a person, even on-line snooping of a series of transactions will not reveal sufficient information to successfully steal the person's identity.

Moreover, because the gene patterns are randomized, no vendor can accumulate sufficient transactions to decode the pattern for any person. With 200 genes and 5 genes per validation, the vendor would have to randomly select 40 unique combinations. This means that the host can monitor a vendor's requests because it will be statistically impossible to have even ten unique combinations on a given person.

There are many uses of this invention that are obvious. For example, any transaction which is not face-to-face and which requires some method of properly identifying one or both parties to the transaction to confirm identity would benefit from this invention. These include
Banking transactions,
Securities transactions,
Picking up children at day-care,
Authenticating permitted use of a computer or workstation,
among others

The following scenario of picking up children at day-care illustrates the use of genetic information to validate a permitted third-party action. In this scenario, the mother has her genetic profile performed and receives both a randomized gene listing and/or an ordered gene listing. She then can select a subset of the genes, say 40, and give that subset listing to the day care provider. The subset listing would ideally be randomized, where the randomization is independent of the mother's randomization. One day it is necessary for her husband, who the day care provider has never met, to pick up the child. Or an uncle or aunt or neighbor. The mother gives the pick-up person the order number and names of, say, 4 genes: i.e., gene 9=PAX, gene 17=VEGF, gene 24=BAS and gene 38=CRC. The pick-up person must correctly provide these four order numbers and the corresponding gene names to be permitted to pick up the child.

The day care center may utilize software to keep track of the gene listings associated with each child. The software can also produce the randomized sub-list queries used to authenticate a permitted pickup. The software can also default into layers or levels of follow-up action in the event invalid authentication occurs, including the possibility of phoning the police for intervention assistance.

The subset listing can be changed at the parent's desire, or on a regular calendar basis. The frequency of changes can be programmed into software or driven by some other means. This ensures the list does not become stale, and also responds to changes in the parent's lives, such as divorce, etc.

Another authenticating use is in corporate or large institutional environments using many computers, where potentially confidential information is at risk to exposure or mis-use. One of the challenges is to ensure that the person logging onto a computer is in fact the person authorized to use that computer. Password protection helps, but is far from secure for many reasons. Use of biomarkers such as fingerprints and virus scans are effective, but often costly to implement.

A more cost effective way of handling the problem is to have the employee provide the employer his/her gene listing (with appropriate privacy considerations and protections). The employer then randomizes the list, and gives the employee a copy of the randomized list. Then, when the employee logs onto a computer or workstation, the final authentication step would be to properly and correctly identify a genetic subset selected from the randomized list. Failure to properly authenticate oneself would automatically lock the keyboard/workstation, and enable further action by the employer. Because this system is software driven and has high flexibility and adaptability, it can be implemented at low cost.

This invention has several advantages over other authenticating methods that utilize random lists of letters and/or numbers, whether alone or in combination. One advantage is that this invention uses information which actually relates to a person's identity—their genes. This makes the information far more relevant and valuable than an arbitrary list of otherwise meaningless letters and/or numbers. This added relevance translates into heightened awareness of the value of this information in establishing identity, and makes it more likely to be both safeguarded by the user and used in real situations.

What is claimed is:

1. A method whereby an enumerated set of genes identified in an individual is utilized by that individual or others to authenticate or validate that individual's identity where such identity authentication or validation is essential to ensure that said individual is the correct party in a transaction that takes place between that individual and any other individual or party.

2. The method of claim 1 wherein the set of genes is a sufficient number to minimize the risk of identity theft.

3. The method of claim 1 wherein the individual can select a subset of genes for use in authenticating an action that is authorized by said individual.

4. The method of claim 1 wherein the set of genes is presented in a non-ordered ‘random’ fashion.

5. A method for identifying an individual comprising
identifying a plurality of genes possessed by an individual,
storing the information about said genes in a first storage medium,
storing the information about said genes in at least one second storage medium,
a means of transferring the information about said genes in a second storage medium to a receiving device,
a means of interpreting the information about said genes in said receiving device,
a means of transferring the information obtained by said receiving device to a second receiving device,
a means of comparing the information received by said second receiving device with the information about said genes in said first storage medium identifiers to determine whether the gene information in the second receiving device and the first storage medium satisfy a predetermined genetic match for one or more criteria relating to the identify of the individual, and
a means of transferring the results of the predetermined genetic match to at least one receiving device,
Wherein the number of genes identified in the individual is at least twenty.

6. The method of claim 5 further comprising a means of displaying the results of the predetermined genetic match.

7. The method of claim 5, further comprising
a means of randomizing the information stored in said first storage device, and
a means of transmitting the information about such randomized ordering to at least one second storage medium.

8. A method for identifying an individual comprising
identifying a plurality of segments of DNA possessed by an individual,
storing the information about said DNA segments in a first storage medium,
storing the information about said DNA segments in at least one second storage medium,
a means of transferring the information about said DNA segments in a second storage medium to a receiving device,
a means of interpreting the information about said DNA segments in said receiving device,
a means of transferring the information obtained by said receiving device to a second receiving device,
a means of comparing the information received by said second receiving device with the information about said DNA segments in said first storage medium identifiers to determine whether the information in the second receiving device and the first storage medium satisfy a predetermined match for one or more criteria relating to the identify of the individual, and
a means of transferring the results of the predetermined DNA match to at least one receiving device,
Wherein the number of DNA segments identified in the individual is at least twenty.

9. The method of claim 8, further comprising
a means of randomizing the information stored in said first storage device, and
a means of transmitting the information about such randomized ordering to at least one second storage medium.

10. The method of claim 8 further comprising a means of displaying the results of the predetermined DNA match.
